(19) United States
(12) Patent Application Publication
(10) Pub. No.: US 2007/0142801 A1
(43) Pub. Date: Jun. 21, 2007

(54) Oil-resistant elastic attachment adhesive and laminates containing it

(76) Inventors: Peiguang Zhou, Appleton, WI (US); Xiaomin Zhang, Appleton, WI (US); Stephen M. Campbell, Winneconne, WI (US)

Correspondence Address: Maxwell J. Petersen;Pauley Petersen & Erickson, Suite 365, 2800 West Higgins Road, Hoffman Estates, IL, 60195, US

(21) Appl. No.: 11/304,193

(22) Filed: Dec. 15, 2005

(51) Int. Cl. A61F 13/15

(52) U.S. Cl. 604/366; 604/372

(57) ABSTRACT

A polyolefin-based elastic hot melt adhesive composition having oil resistance, creep resistance and excellent bond strength is provided. The adhesive composition is useful in elastic laminates used in personal care absorbent articles and other garments.

### BACKGROUND OF THE INVENTION

This invention relates to an oil-resistant elastic attachment adhesive and laminates containing it.

Attachment adhesives are used in personal care absorbent articles for bonding elastic strands such as spandex (polyurethane) strands to a polyolefin film or nonwoven substrate. The laminates of elastic strands bonded to a substrate can be used to form waist elastic bands, leg elastic bands and other elastic components of the article. Typically, the elastic strands are bonded to the substrate using a hot melt adhesive while the elastic strands are in a stretched (tensioned) condition and the substrate is relaxed.

Various elastic attachment adhesives perform suitably during manufacture and subsequent storage of the absorbent article. However, problems have arisen when the absorbent article is donned on a wearer who is wearing an oil-containing lotion. The oil may interfere with the bonding, and cause delamination of the elastic strands from the substrate and loss of elastic function.

Because oil-based lotions are used frequently by wearers of absorbent articles, there is a need or desire for elastic attachment adhesives and corresponding laminates which resist the oil-based lotions and maintain their adhesion. Such adhesives should provide suitable bonding to spandex (polyurethane) strands and to a polyolefin film or nonwoven web, while the strands are in a stretched condition. Such adhesives should also be relatively inexpensive. For instance, polyolefin-based elastic adhesives are typically less expensive than other elastic adhesives based on various rubbers.

### SUMMARY OF THE INVENTION

The present invention is directed to an elastic attachment adhesive including a single-site catalyzed polyolefin-based plastomer having a number average molecular weight of about 1,000 to about 50,000 grams/mol and a crystallinity of 10 to less than 50% by weight, a crystalline propylene-based polymer having a molecular weight greater than 10,000 to about 300,000 grams/mol and a crystallinity of 50-100% by weight, a tackifier, and an elastomer. The elastic attachment adhesive provides a bond between spandex (polyurethane) strands and a polyolefin substrate that is resistant to mineral oil.

The present invention is also directed to an elastic laminate including a first facing layer, a second facing layer, and the foregoing elastic attachment adhesive bonding the first facing layer to the second facing layer.

The present invention is also directed to a personal care absorbent article including a chassis defining a waist opening and two leg openings, side panels extending laterally from the chassis, a waist region adjacent to the waist opening and leg regions adjacent to the leg openings. The chassis includes a substantially liquid impermeable outer cover, a liquid permeable bodyside liner, and an absorbent core between the outer cover and the bodyside liner. At least one of the outer cover, side panels, waist region and leg regions includes an elastic laminate of the invention.

With the foregoing in mind, it is a feature and advantage of the invention to provide an improved elastic attachment adhesive having excellent bonding and oil resistance, an elastic laminate which utilizes the elastic attachment adhesive between two facing layers, and a personal care absorbent article which utilizes the elastic laminate in one or more components of the article. The foregoing and other features and advantages will become further apparent from the following detailed description of the invention, and the accompanying drawings.

### BRIEF DESCRIPTION OF THE DRAWINGS

FIG. 1 is a plan view of one embodiment of a laminate including the adhesive composition of the invention.

FIG. 2 is a sectional view of another embodiment of a laminate including the adhesive composition of the invention.

FIG. 3 is a plan view of a personal care absorbent article including the laminate of the invention.

FIG. 4 is a schematic illustration of the creeping resistance test procedure described below.

### DEFINITIONS

“Bonded” refers to the joining, adhering, connecting, attaching, or the like, of at least two elements. Two elements will be considered to be bonded together when they are bonded directly to one another or indirectly to one another, such as when each is directly bonded to intermediate elements.

“Conventional hot-melt adhesive” means a formulation that generally comprises several components. These components typically include one or more polymers to provide cohesive strength (e.g., aliphatic polyolefins such as poly (ethylene-co-propylene) copolymer; ethylene vinyl acetate copolymers; styrene-butadiene or styrene-isoprene block copolymers; etc.); a resin or analogous material (sometimes called a tackifier) to provide adhesion (e.g., hydrocarbons distilled from petroleum distillates; rosins and/or rosin esters; terpenes derived, for example, from wood or citrus, etc.); perhaps waxes, plasticizers or other materials to modify viscosity (i.e., flowability) (examples of such materials include, but are not limited to, mineral oil, polybutene, paraffin oils, ester oils, and the like); and/or other additives including, but not limited to, antioxidants or other stabilizers. A typical hot-melt adhesive formulation might contain from about 15 to about 35 weight percent cohesive strength polymer or polymers; from about 50 to about 65 weight percent resin or other tackifier or tackifiers; from more than zero to about 30 weight percent plasticizer or other viscosity modifier; and optionally less than about 1 weight percent stabilizer or other additive. It should be understood that other adhesive formulations comprising different weight percentages of these components are possible.

“Elastic tension” refers to the amount of force per unit width required to stretch an elastic material (or a selected zone thereof) to a given percent elongation.

“Elastomeric” and “elastic” are used interchangeably to refer to a material or composite that is generally capable of recovering its shape after deformation when the deforming force is removed. Specifically, as used herein, elastic or elastomeric is meant to be that property of any material which, upon application of a biasing force, permits the material to be stretchable to a stretched biased length which is at least about 50 percent greater than its relaxed unbiased length, and that will cause the material to recover at least 40 percent of its elongation upon release of the stretching force. A hypothetical example which would satisfy this definition of an elastomeric material would be a ten (10) cm sample of a material which is elongatable to at least 15 cm and which, upon being elongated to 15 cm and released, will recover to a length of less than 13 cm. Many elastic materials may be stretched by much more than 50 percent of their relaxed length, and many of these will recover to substantially their original relaxed length upon release of the stretching force.

“Elongation” refers to the capability of an elastic material to be stretched a certain distance, such that greater elongation refers to an elastic material capable of being stretched a greater distance than an elastic material having lower elongation.

“Film” refers to a thermoplastic film made using a film extrusion process, such as a cast film or blown film extrusion process. The term includes apertured films, slit films, and other porous films which constitute liquid transfer films, as well as films which do not transfer liquid.

“Garment” includes personal care absorbent articles, medical garments, industrial workwear garments, and the like. The term “disposable garment” includes garments which are typically disposed of after 1-5 uses. The term “personal care absorbent article” includes diapers, training pants, swim wear, absorbent underpants, adult incontinence products, feminine hygiene products, and the like. The term “medical garment” includes medical (i.e., protective and/or surgical) gowns, caps, gloves, drapes, face masks, and the like. The term “industrial workwear garment” includes laboratory coats, cover-alls, and the like.

“Hot-melt processable” means that an adhesive composition may be liquefied using a hot-melt tank (i.e., a system in which the composition is heated so that it is substantially in liquid form) and transported via a pump (e.g., a gear pump or positive-displacement pump) from the tank to the point of application proximate to a substrate or other material; or to another tank, system, or unit operation (e.g., a separate system, which may include an additional pump or pumps, for delivering the adhesive to the point of application). Hot-melt tanks used to substantially liquefy a hot-melt adhesive typically operate in a range from about 200 degrees Fahrenheit to about 400 degrees Fahrenheit. Generally, at the point of application, the substantially liquefied adhesive composition will pass through a nozzle or bank of nozzles, but may pass through some other mechanical element such as a slot. A hot-melt processable adhesive composition is to be contrasted with a composition that requires a conventional extruder, and the attendant pressures and temperatures characteristic of an extruder, to liquefy, mix, and/or convey the composition. While a hot-melt tank and pump in a hot-melt processing system can handle adhesive-composition viscosities in a range of up to about 50,000 centipoise, an extruder can handle and process adhesive-composition viscosities in a range from about 10,000 centipoise to viscosities of several hundred thousand centipoise.

“Layer” when used in the singular can have the dual meaning of a single element or a plurality of elements.

“Low softening point additive” refers to a tackifier or wax or low molecular weight polymers having a softening point below 80 degrees Celsius, and a viscosity of less than 1000 cps at 182 degrees Celsius as measured by a ring and ball method (ASTM E-28). “High softening point” additive refers to tackifiers and the like having softening points of 80° C. or higher, suitably 90° C. or higher, or 100° C. or higher, and up to about 150° C.

“Meltblown fiber” refers to fibers formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface.

“Nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Softening point” refers to a material softening temperature, typically measured by a ring and ball type method, ASTM E-28.

“Spunbond fiber” refers to small diameter fibers which are formed by extruding molten thermoplastic material as filaments from a plurality of fine capillaries of a spinnerette having a circular or other configuration, with the diameter of the extruded filaments then being rapidly reduced as taught, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein by reference in its entirety in a manner consistent with the present document. Spunbond fibers are quenched and generally not tacky when they are deposited onto a collecting surface. Spunbond fibers are generally continuous and often have average deniers larger than about 0.3, more particularly, between about 0.6 and 10.

“Strand” refers to an article of manufacture whose width is less than a film and is suitable for incorporating into a film, according to the present invention.

“Thermoplastic” describes a material that softens and flows when exposed to heat and which substantially returns to a nonsoftened condition when cooled to room temperature.

“Vertical filament stretch-bonded laminate” or “VF SBL” refers to a stretch-bonded laminate made using a continuous vertical filament process, as described herein.

“Woven” fabric or web means a fabric or web containing a structure of fibers, filaments, or yarns, which are arranged in an orderly, inter-engaged fashion. Woven fabrics typically contain inter-engaged fibers in a “warp” and “fill” direction. The warp direction corresponds to the length of the fabric while the fill direction corresponds to the width of the fabric. Woven fabrics can be made, for example, on a variety of looms including, but not limited to, shuttle looms, rapier looms, projectile looms, air jet looms, and water jet looms.

These terms may be defined with additional language in the remaining portions of the specification.

### DETAILED DESCRIPTION OF THE INVENTION

In accordance with the invention, an elastic adhesive composition includes a single-site catalyzed (e.g., metallocene-catalyzed or constrained geometry-catalyzed) polyolefin-based plastomer having a number average molecular weight of about 1,000 to about 50,000 grams/mol and a crystallinity of 10 to less than 50% by weight, a crystalline propylene-based polymer having a number average molecular weight of greater than 10,000 to about 200,000 grams/mol and a crystallinity of 50-100% by weight, a tackifier, and an elastomer.

The single-site catalyzed polyolefin-based plastomer is suitably present at about 20-70% by weight, or about 25-60% by weight, or about 30-50% by weight of the adhesive composition. The plastomer can be a polyolefin homopolymer or copolymer, and is suitably a copolymer. The copolymer can include about 5-85% by weight, or about 10-50% by weight, or about 15-35% by weight of a C₃-C₁₂ alpha-olefin comonomer, and about 15-95% by weight, or about 50-90% by weight, or about 65-85% by weight ethylene. Particularly suitable comonomers are hexene and octene. The plastomer may alternatively include propylene or another alpha-olefin as the major comonomer component, and ethylene or another alpha olefin as the minor comonomer component. The plastomer may suitably have a number average molecular weight of about 1,000 to about 50,000 grams/mol, or about 2,000 to about 30,000 grams/mol, or about 3,000 to about 20,000 grams/mol, and may suitably have a crystallinity of about 12-40% by weight, or about 15-30% by weight. The plastomer may be produced using any single-site (e.g., metallocene or constrained geometry) catalyst which yields a polyolefin-based plastomer with the foregoing properties. The plastomer may have a Brookfield viscosity at 182° C. of at least about 3,000 centipoise, suitably about 5,000-25,000 centipoise, or about 10,000-20,000 centipoise. The plastomer may have a molecular weight distribution (defined as a ratio of weight average molecular weight to number average molecular weight) of about 1.5-4.0, suitably about 2.0-3.5. The plastomer may have a melt index (190° C.) of about 100-2000 grams/10 min or about 300-1200 grams/10 min, and a density of about 0.850-0.890 grams/cm³, suitably about 0.860-0.880 grams/cm³. The plastomer may have a glass transition temperature of less than −10° C. or less than −30° C., or less than −50° C. Suitable polyolefin-based plastomers are produced by Dow Chemical Co. under the trade name AFFINITY®, and include without limitation AFFINITY® GA1900 and GA1950.

The crystalline propylene-based polymer is suitably present at about 5-35% by weight, or about 8-30% by weight, or about 10-20% by weight of the adhesive composition. This polymer can be a propylene homopolymer or a copolymer of propylene with ethylene or a C₄-C₁₂ alpha-olefin comonomer, and may contain up to about 10% by weight of the comonomer. Particularly suitable propylene-based polymers are polypropylene homopolymers, and block or random copolymers of propylene with ethylene or butene. The crystalline propylene-based polymer may suitably have a number average molecular weight of about 15,000-150,000 grams/mol, or about 20,000-100,000 grams/mol, and may suitably have a crystallinity of about 55-90% by weight, or about 60-85% by weight. The crystalline propylene-based polymer may be produced using any catalyst (e.g. single-site or Ziegler-Natta) which yields a polymer having the foregoing properties.

The tackifier is suitably present at about 15-60% by weight of the adhesive composition, or about 25-55% by weight, or about 35-55% by weight. Suitably, the tackifier is a high softening point tackifier having a softening point of 80° C. or higher, or 100° C. or higher. Examples include without limitation aliphatic and aromatic hydrocarbon tackifiers sold by ExxonMobil Chemical Co. under the trade name ESCOREZ®. Of these ESCOREZ® 5000-series tackifiers typically have softening points above 80° C. Additionally, H-100 series tackifiers sold by Eastman Chemical Co. typically have high softening points.

Alternatively, the tackifier may be a tackifier blend. The tackifier blend may contain about 50-75%, or about 60-70% by weight of the tackifier blend, of a tackifier having a softening point of at least 90° C. and a glass transition temperature of at least 50° C. and about 25-50%, or about 30-40% by weight of the tackifier blend, of a tackifier having a softening point less than 50° C. and a glass transition temperature less than 30° C. ESCOREZ® 2500 tackifier has a softening point of about 20° C. and a glass transition temperature of −15° C.

The elastomer is suitably present at about 2-20% by weight of the adhesive composition, or about 3-10% by weight, or about 4-8% by weight. Suitable elastomers include without limitation styrene-based block copolymer elastomers sold under the trade name KRATON® by Kraton Polymers LLC and those sold under the trade name SEPTON® by Septon Company America. Examples include styrene-isoprene diblock copolymers, styrene-butadiene diblock copolymers, styrene-isoprene-styrene triblock copolymers, styrene-butadiene-styrene triblock copolymers, styrene-(ethylene-butene)-styrene triblock copolymers, styrene-(ethylene-propylene)-styrene triblock copolymers, styrene-(ethylene-butene)-styrene-(ethylene-butene) tetrablock copolymes, styrene (ethylene-propylene)-styrene-(ethylene-propylene) tetrablock copolymers, and combinations thereof. Other suitable elastomers include without limitation polyisoprene, polybutadienes, ethylene vinyl acetate copolymers, ethylene methyl acrylate copolymers, ethylene methyl methacrylate copolymers, ethylene ethyl acrylate copolymers, ethylene n-butyl acrylate copolymers, single-site catalyzed elastomeric copolymers of ethylene and a C₄-C₁₂ alpha olefin comonomer, and combinations thereof.

The elastic adhesive composition may also include other ingredients and additives provided that the four components described above remain within stated percentage ranges. Other ingredients and additives may include without limitation antioxidants, plasticizers, mineral oil, pigments, filler, polymer compatibilizers and combinations thereof. These other ingredients and additives may collectively constitute about 0.1-25% by weight of the adhesive composition, suitably about 0.5-15% by weight, or about 1-10% by weight.

The adhesive composition of the invention is hot melt processable, and may be applied to a substrate using any technique suitable for applying hot melt adhesives. Examples of suitable hot melt processing techniques are described in U.S. Patent Application Publication 2005/0054779A1, published on 10 Mar. 2005, the disclosure of which is incorporated by reference. The adhesive composition suitably has a viscosity of about 1,000 to 10,000 cps at temperatures between 150° C. and 185° C.

Laminates can be formed using the adhesive compositions to bond together two layers of nonwoven material, woven material, hook material, film, or other facing materials, or elasticized components such as elastic strands. The facing materials themselves may be laminates, such as necked-bonded laminates. Laminates including the adhesive compositions of the invention have significant temperature resistance, oil resistance and stretch capabilities compared to laminates including conventional adhesives.

Because of the stretchable properties of the adhesive composition, the adhesive composition is particularly suitable for bonding stretchable or elastomeric layers or components to one another. Therefore, stretchable facing layers, such as necked-bonded laminates (NBL), stretch-bonded laminates (SBL), point unbonded materials, and hook material as used in hook-and-loop fasteners, can be successfully bonded using the adhesive composition of the invention. For additional detail on how NBLs and other neck-bonded materials are formed, see U.S. Pat. No. 5,336,545 to Morman, entitled “Composite Elastic Necked-Bonded Material,” which is hereby incorporated by reference in its entirety in a manner consistent with the present document. An SBL is generally a laminate made up of an elongated elastic web or elongated elastomeric strands bonded between two spunbond layers, for example. For additional detail on how SBLs are formed, see European Patent Application No. EP 0 217 032 published on Apr. 8, 1987 in the names of Taylor et al., which is hereby incorporated by reference in its entirety in a manner consistent with the present document. Point unbonded materials are fabrics having continuous thermally bonded areas defining a plurality of discrete unbonded areas and are described in greater detail in U.S. Pat. No. 5,858,515 issued Jan. 12, 1999 to Stokes, et al., hereby incorporated by reference in its entirety in a manner consistent with the present document. Hook material typically includes a base or backing structure and a plurality of hook members extending outwardly from at least one surface of the backing structure. In contrast to loop material, which is typically a flexible fabric, hook material advantageously includes a resilient material to minimize unintentional disengagement of the hook members as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene, or other suitable material. Examples of commercially available hook material are available from Velcro Industries B.V., Amsterdam, Netherlands or affiliates thereof, as well as from Minnesota Mining & Manufacturing Co., St. Paul, Minn., U.S.A.

FIG. 1 is a plan view of a laminate 10 including a substrate 12 combined with a plurality of elastic strands 14, using an adhesive layer 16 formed from the adhesive composition of the invention. Typically, the elastic strands 14 are bonded to the substrate 12 while the elastic strands 14 are in a stretched state, using a conventional method. The illustrated laminate 10 is useful for forming elastic waistbands used in the waist region of a personal care absorbent article, and/or elastic leg bands used in the leg regions of the personal care absorbent article. When the laminate 10 is permitted to relax, the elastic waist bands and/or leg bands retract and may assume a puckered or ruffled configuration, causing the personal care absorbent article to fit snugly about a wearer.

The elastic strands 14 may be formed of spandex (i.e., segmented polyurethane) strands, such as LYCRA® strands available from E.I. DuPont DeNemours & Co. The elastic strands 14 may also be formed of styrene-based block copolymers, including any of the KRATON® or SEPTON® polymers identified above as being useful for the elastomeric component of the adhesive composition. Other types of elastic polymers can also be employed to form the elastic strands.

The substrate 12 can be a thermoplastic film, nonwoven fibrous web, fibrous web, or a combination thereof. When the substrate 12 is a thermoplastic film or fibrous nonwoven web, it can be formed from any suitable thermoplastic polymer. Examples include polyethylene homopolymers, ethylene-alpha olefin copolymers, polypropylene homopolymers, propylene-ethylene and propylene-alpha olefin copolymers, polyamides, polyurethanes, polyesters and the like. When the substrate 12 is a nonwoven web, it may be a spunbond web, meltblown web, bonded carded web, airlaid web, or combination of more than one nonwoven layer. The substrate 12 may also be a combination of a film and a nonwoven web.

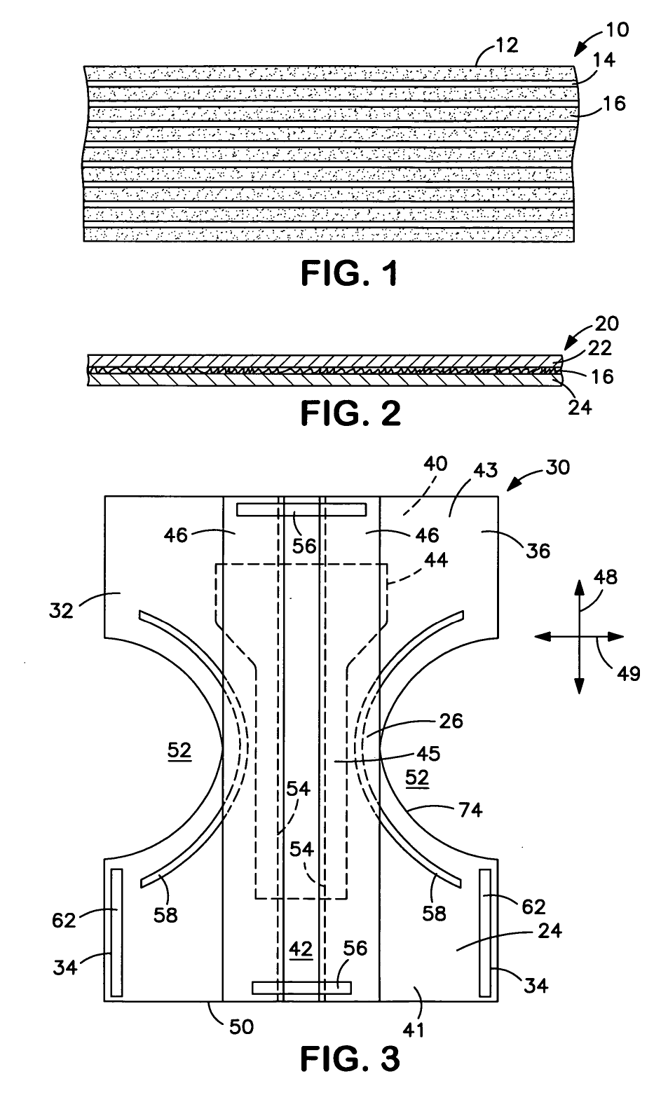

FIG. 2 is a sectional view of a laminate 20 including a first facing layer 22, a second facing layer 24, and an adhesive layer 16 of the invention bonding the facing layers together. In the illustrated embodiment, the facing layer 22 may be a thermoplastic film, and may be a breathable, stretch-thinned film formed by combining polyolefin and a filler, and stretching the film formed from the combination to form micropores or voids around the filler particles. The facing layer 24, which is laminated to the film, may be a fibrous nonwoven web, such as a spunbond web. This embodiment of laminate 20 is useful as a liquid-impermeable outercover for a personal care absorbent article.

Alternatively, facing layers 22 and 24 may both be fibrous nonwoven webs. This embodiment of laminate 20 is useful for forming side panels on personal care absorbent articles. Alternatively, facing layers 22 and 24 may be two films, or a film/nonwoven laminate combined with a film or nonwoven web, or any other suitable substrate combination. In one embodiment, one of the facing layers 24 is a hook-type fastener material and the other is a spunbond/film/spunbond neck-bonded laminate used to make stretchable side panels. The combination of the side panel material with the hook fastener material provides a hook fastener tab which can be releasably joined to an opposing loop fastener material.

The adhesive composition of the invention provides an oil-resistant bond to mineral oil. To measure oil resistance, a laminate made using the adhesive composition is soaked in mineral oil for 72 hours at room temperature. The adhesive bond strength between the first facing layer and the second facing layer is measured before and after immersion of the laminate in mineral oil. Alternatively, the adhesive bond strength is measured using laminate samples which are not immersed in mineral oil and samples which are immersed for 72 hours. Suitably, the adhesive bond strengths are measured using the Dynamic Peel Test described below. An average of five sample measurements is used to determine each adhesion value. If the adhesion falls by less than 30% due to the immersion in mineral oil, the laminate is considered oil-resistant. Suitably, the adhesion may fall by less than 20%, or less than 10%, or about 0%, due to the immersion in mineral oil.

FIG. 3 is a plan view of a personal care absorbent article 30, which is illustrated as a diaper. The diaper 30 includes an absorbent chassis 32 defining a front waist region 41, a back waist region 43, and a crotch region 45 interconnecting the front and back waist regions. In the fastened position, the absorbent chassis 32 defines a three-dimensional pant configuration having a waist opening 50 and a pair of leg openings 52. The chassis 32 includes a body side liner 42 which is configured to contact the wearer, and an outer cover 40 opposite and generally coextensive with the body side liner which is configured to contact the wearer's clothing. An absorbent structure 44 is positioned or located between the outer cover 40 and the body side liner 42. The absorbent structure 44 has a shorter length and width than the outer cover 40 and body side liner 42 and is spaced inboard from all edges of the outer cover 40 and body side liner 42.

The absorbent structure 44 can be any structure which is generally compressible, conformable, non-irritating to the wearer's skin, and capable of absorbing and retaining liquids and certain body wastes at anticipated levels despite the narrowed crotch width. The absorbent structure 44 can be manufactured in a wide variety of sizes and shapes, and from a wide variety of liquid absorbent materials commonly used in the art. For example, the absorbent structure 44 can suitably include a matrix of hydrophilic fibers, such as a web of cellulosic fluff, mixed with particles of a high-absorbency material commonly known as superabsorbent material. In certain embodiments, the absorbent structure 44 includes a matrix of cellulosic fluff, such as wood pulp fluff, and superabsorbent hydrogel-forming particles. The wood pulp fluff can be exchanged with synthetic, polymeric, meltblown fibers or with a combination of meltblown fibers and natural fibers. The superabsorbent particles can be substantially homogeneously mixed with the hydrophilic fibers or can be nonuniformly mixed. The fluff and superabsorbent particles can also be selectively placed into desired zones of the absorbent structure 44 to better contain and absorb body exudates. The absorbent structure 44 can have variable thickness, with greater thickness in “target” areas, such as in a central portion of the crotch region. The concentration of the superabsorbent particles can also vary through the thickness of the absorbent structure 44. Alternatively, the absorbent structure 44 can include a laminate of fibrous webs and superabsorbent material or other suitable means of maintaining a superabsorbent material in a localized area. The absorbent structure 44 may or may not be wrapped or encompassed by a suitable tissue wrap that maintains the integrity and/or shape of the absorbent structure 44.

The outer cover 40 of the diaper 30 suitably includes a material that may be substantially liquid impermeable or liquid permeable, and can be elastic, stretchable, extensible, non-stretchable, or non-extensible. The outer cover 40 can be a single layer of liquid impermeable material, but suitably includes a multi-layered laminate structure in which at least one of the layers is liquid impermeable. For instance, the outer cover 40 can include a liquid permeable outer layer and a liquid impermeable inner layer that are suitablyjoined together by a laminate adhesive (not shown).

The inner layer of the outer cover 40 can be both liquid and vapor impermeable, or can be liquid impermeable and vapor permeable. The inner layer of the outer cover 40 desirably includes a material that can be elastic, stretchable, extensible, non-stretchable, or non-extensible. The inner layer is desirably manufactured from a thin plastic film, although other flexible liquid impermeable materials may also be used. The inner layer, or the liquid impermeable outer cover 40 when a single layer, prevents waste material from wetting articles, such as bedsheets and clothing, as well as the wearer and care giver. A suitable liquid impermeable film for use as a liquid impermeable inner layer, or a single layer liquid impermeable outer cover 40, is a 0.02 millimeter polyethylene film commercially available from Pliant Corporation of Schaumburg, Ill., U.S.A. If the outer cover 40 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 40. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn.

Certain “non-breathable” elastic films can also be used to make the outer cover 40. Examples of suitable non-breathable films can be made of styrene-ethylene-butylene-styrene or styrene-isoprene-styrene block copolymers, KRATON® polymers from Kraton Inc. of Houston, Tex., U.S.A., metallocene catalyzed elastomers or plastomers, and the like. Other materials suitable for making the outer cover 40 include monolithic breathable films, such as those made of polyether amide based polymers, for example PEBAX, and ether/ester polyurethane thermoplastic elastomers.

In one embodiment, the laminate 20 shown in FIG. 2 can be employed as an outer cover 40 in a personal care absorbent article. By employing the elastic adhesive composition of the invention as adhesive layer 16, the bond between the inner and outer layers of the outer cover 40 can be rendered oil-resistant. Also, if the outer cover 40 is designed to be stretchable or elastic, the adhesive layer 16 will retain its bonding during any stretching and recovery of the outer cover 40 when the diaper is in use.

The liquid permeable body side liner 42 is illustrated as overlying the outer cover 40 and absorbent structure 44, and may but need not have the same dimensions as the outer cover 40. The body side liner 42 is desirably compliant, soft feeling, and non-irritating to the wearer's skin. Further, the body side liner 42 can be less hydrophilic than the absorbent structure 44, to present a relatively dry surface to the wearer and permit liquid to readily penetrate through its thickness. The body side liner 42 desirably includes a material that can be elastic, stretchable, extensible, non-stretchable, or non-extensible.

The body side liner 42 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics can be used for the body side liner 42. For example, the body side liner can be composed of a meltblown or spunbonded web of polyolefin fibers. The body side liner can also be a bonded-carded web composed of natural and/or synthetic fibers. The body side liner can be composed of a substantially hydrophobic material, and the hydrophobic material can, optionally, be treated with a surfactant or otherwise processed to impart a desired level of wettability and hydrophilicity.

The absorbent chassis 32 may further include a pair of transversely opposed side panels or tabs 34, which extend transversely outward along the back waist region 24 of the absorbent chassis 32. The side panels 34 may be integrally formed with the outer cover 40 and/or the body side liner 42, or may include two or more separate elements.

In one embodiment, the side panels 34 may be formed using a laminate 20 of the invention as shown in FIG. 2. For example, the side panels 34 may be formed of an elastic or stretchable film laminated to a laterally stretchable nonwoven web, such as a necked spunbond web, or laminated between two laterally stretchable nonwoven webs. Use of the elastic adhesive composition of the invention to bond the layers together provides the laminate with oil resistance, and with a strong bond that is maintained during any stretching and recovery of the laminate while the diaper is in use.

The diaper 30 may also include a waist elastic member 56 in the front waist region 22, in the back waist region 24, or in both the front and back waist regions 22, 24 of the garment, operatively attached to the outer cover 40 and/or body side liner 42 and extending across part or a full length of the waist regions.

To further enhance containment and/or absorption of body exudates, the diaper 30 may also include leg elastic members 58, as are known to those skilled in the art. The leg elastic members 58 may be operatively joined to the outer cover 40 and/or body side liner 42 along opposite side edges of the absorbent chassis 32 and positioned in the crotch region 26 of the diaper 30.

The waist elastic members 56 and the leg elastic members 58 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat; such that elastic constrictive forces are imparted to the substrate. In one particular embodiment, for example, the waist elastic members 56 may include a styrene-(ethylene-propylene)-styrene (SEPS) block copolymer, such as KRATON® G2760, available from Kraton Inc. of Houston, Tex., U.S.A. Segmented polyurethane strands formed of LYCRA® are also useful.

In useful embodiments, the waist elastic members and/or leg elastic members can be formed using a laminate 10 of the invention as shown in FIG. 1. The elastic strands 14 may be bonded, while in a stretched state, to a film or nonwoven web substrate 12 which subsequently gathers when the elastic strands 14 retract. Use of the adhesive composition of the invention as adhesive layer 16 provides the waist and/or leg elastics with oil resistance, and provides a strong adhesive bond which is maintained during stretching and recovery of the elastic members.

The diaper 30 may be refastenable, thereby including a refastenable fastening system for securing the diaper about the waist of the wearer. One example of a suitable refastenable fastening system may include fastening components 62, such as hook components, located along or adjacent to distal edges of the side panels 34. Suitable single-sided hook materials are available from Velcro Industries B.V., Amsterdam, Netherlands, or affiliates thereof. The fastening components 62 are adapted to refastenably connect to mating fastening components, such as loop material, located on an outer surface of the front waist region 41. One example of suitable loop material is “point unbonded” material. Point unbonded materials are fabrics having continuous thermally bonded areas defining a plurality of discrete unbonded areas and are described in greater detail in U.S. Pat. No. 5,858,515 issued Jan. 12, 1999 to Stokes, et al., incorporated herein by reference. The engaging elements of the fastening components 62 are adapted to repeatedly engage and disengage the engaging elements of the mating fastening components.

In one useful embodiment, the male fastening components 62 may be secured to the side panels 34 using the adhesive composition of the invention. This is particularly suitable when the ear panels 34 are formed of a stretchable or elastic material, such as a neck-bonded laminate wherein a stretchable or elastic film is bonded between two neck-stretched spunbond webs. By employing the elastic adhesive composition of the invention, delamination of the fastening components 62 from the side panels 34 due to stretching of the side panels can be prevented.

To enhance containment and/or absorption of any body exudates discharged from the wearer, the chassis 32 may include a pair of containment flaps 46 which are configured to provide a barrier to the transverse flow of body exudates. A flap elastic member 54 may be operatively joined with each containment flap 46 in any suitable manner as is well known in the art. The elasticized containment flaps 46 define an unattached edge which assumes an upright, generally perpendicular configuration in at least the crotch region 26 of the diaper 30 to form a seal against the wearer's body. The containment flaps 46 can be located along the transversely opposed side edges of the chassis 32, or in the crotch region 26, and can extend longitudinally along the entire length of the chassis or may only extend partially along the length of the chassis. Suitable constructions and arrangements for the containment flaps 46 are generally well known to those skilled in the art.

In useful embodiments, the elasticized containment flaps 46 can be formed using a laminate 10 of the invention as illustrated in FIG. 1. The elastic strands 14 may be bonded, while in the stretched state, to a film or nonwoven web substrate which subsequently gathers when the elastic strands 14 retract. Use of the adhesive composition of the invention as adhesive layer 16 provides the containment flaps 46 with oil resistance, and provides a strong adhesive bond which is maintained during stretching and recovery of the containment flaps.

The components of the personal care absorbent article 30 can be assembled and joined together using conventional techniques. While the personal care absorbent article is illustrated as a diaper, the adhesive composition and the laminate 10 or 20 of the invention can be employed in a wide variety of personal care absorbent articles having similar or related structures. Examples include training pants, adult incontinence articles, absorbent swim wear, absorbent underpants, feminine hygiene products and the like. The adhesive composition and laminates of the invention can also be used in other types of garments, such as medical garments and industrial workwear garments, in appropriate situations.

### EXAMPLES

Adhesive compositions were formulated from four base polymers. The base polymers were:

The four base polymers had the following properties.

TABLE 1
Base Polymer Properties

| Base | Viscosity | Soft Pt. | | Tg | | | Tensile |
|---|---|---|---|---|---|---|---|
| Polymer | Cps | ° C. | Crystallinity % | ° C. | Elongation % | MWD | N/cm² |
| A | 2200 (177° C.) | 152 | 15 | −20 | — | 5-10 | 230 |
| B | 1500 (190° C.) | 110 | 3 | −23 | — | 5-10 | 38 |
| C | 2300 (190° C.) | 155 | — | −10 | 100 | 4-6 | 138 |
| D | 17000 (177° C.) | <70 | 18.3 | −57 | 150 | 2.0 | 235 |

A REXTAC ® 2115, available from Huntsman Polymers, which is an amorphous polypropylene;
B REXTAC ® 2715, available from Huntsman Polymers, which is an amorphous propylene-butene-1 copolymer;
C PP1023, available from Eastman Chemical Co., which is an amorphous polypropylene; and
D AFFINITY ® GA-1950, available from Dow Chemical Co., which is a single-site catalyzed polyolefin plastomer (POP).

The properties were measured as follows:

| | |
|---|---|
| Viscosity: | Test described below. |
| Softening Point: | Ring and Ball Method, ASTM E-28. |
| Crystallinity: | ASTM D3418-03 (Differential scanning |
| | calorimetry) |
| Glass Transition: | ASTM D3418-03 |
| % Elongation: | Test described below. |
| Mol. Wt. Distribution: | Test described below. |
| Tensile Force: | Tension force test (described below). |

The four base polymers were formulated into hot melt adhesives using the following tackifiers, isotactic (crystalline) polypropylenes, elastomers and stabilizer.

Tackifiers

| | | Ring & | | Brookfield | |
|---|---|---|---|---|---|
| | | Ball | | Viscosity, | |
| | | Soft Point | | 177° C. | Manu- |
| | Tackifier | (° C.) | Tg ° C. | cps | facturer |
| A | Escorez 2520 | 20 | −15 | — | ExxonMobil |
| B | Escorez 5300 | 105 | 55 | 300 | ExxonMobil |
| C | Escorez 5320 | 122 | 65 | 1500 | ExxonMobil |
| D | Escorez 5380 | 85 | 35 | 100 | ExxonMobil |
| E | Escorez 5600 | 103 | 55 | 300 | ExxonMobil |
| F | Escorez 5690 | 89 | 45 | 100 | ExxonMobil |
| G | H-100R | 100 | — | 200 (190° C.) | Eastman |
| | | | | | Chemical |
| H | H-130R | 130 | — | 1200 (190° C.) | Eastman |
| | | | | | Chemical |

Isotactic (Crystalline) Polypropylenes

| | | | Melt Flow Rate | |
|---|---|---|---|---|
| | | | (230° C., | |
| | Polypropylene | Type | grams/10 min.) | Manufacturer |
| A | CP15000P | Homopolymer | 1200 | Sunoco |
| B | PF-017 | Homopolymer | 1700 | Basell |
| C | PP3746G | Homopolymer | 1500 | ExxonMobil |

Elastomers

| | Elastomer | Type | Manufacturer |
|---|---|---|---|
| A | MD6932 | Styrene-(ethylene-propylene)- | Kraton Polymers LLC |
| | | styrene triblock | |
| B | 2002 | Styrene-(ethylene-propylene)- | Septon Company |
| | | styrene triblock | America |

Stabilizer
IRGANOX® 1010, from Ciba Specialty Chemical Co.

The hot melt adhesive formulations are set forth in Table 2 (below). Adhesives SA-16, SA-19 and SA-25 are controls. The remaining formulations are inventive.

TABLE 2
Adhesive Composition Formulations

| | Brookfield | | | | | |
|---|---|---|---|---|---|---|
| Adhesive | Viscosity Cps | Base polymer | | | | Irganox |
| Code | (182° C.) | wt % | Tackifier % | Isotactic PP % | Elastomer % | 1010, (%) |
| SA-16 | 4880 | 48.5% | 30% Escorez | 16% Sunoco | 6% Kraton | 0.50% |
| | | RT2715 | 5600 | CP-15000p | MD 6932 | |
| SA-19 | 2680 | 50% RT2115 | 30% Escorez | 16% Basell | 4% Septon 2002 | 0.50% |
| | | | 5690 | PF-017 | | |
| SA-24 | 3740 | 30% GA-1950 | 49.5% Escorez | 15% PP3746G | 5% Septon 2002 | 0.50% |
| | | | 5300 | | | |
| SA-25 | 3360 | 49.5% PP1023 | 30% H-100R | 15% PP3746G | 5% Septon 2002 | 0.50% |
| SA-26 | 3050 | 30% GA-1950 | 49.5% Escorez | 15% PP3746G | 5% Septon 2002 | 0.50% |
| | | | 5380 | | | |
| SA-27 | 3890 | 30% GA-1950 | 49.5% H-100R | 15% PP3746G | 5% Septon 2002 | 0.50% |
| SA-28 | 4660 | 29.5% GA- | 50% Escorez | 15% PP3746G | 5% Septon 2002 | 0.50% |
| | | 1950 | 5320 | | | |
| SA-29 | 4130 | 29.5% GA- | 50% Escorez | 15% PP3746G | 5% Septon 2002 | 0.50% |
| | | 1950 | 5600 | | | |
| SA-30 | 3550 | 29.5% GA- | 50% Escorez | 15% PP3746G | 5% Septon 2002 | 0.50% |
| | | 1950 | 5690 | | | |
| SA-31 | 4610 | 29.5% GA- | 50% H-130R | 15% PP3746G | 5% Septon 2002 | 0.50% |
| | | 1950 | 35% H-130R+ | | | |
| SA-33 | 3880 | 29.5% GA- | 15% Escorez | 15% PP3746G | 5% Septon 2002 | 0.50% |
| | | 1950 | 2520 | | | |

Using controlled meltblowing bonding conditions, many of the foregoing adhesives were tested along with FINDLEY H2840 from Findley Adhesives, Inc. to prepare laminates of LYCRA® strands to a polypropylene spunbond web having a basis weight of 17 grams/m². The laminates were prepared according to the Creep Resistance Test (below), and were elongated 250% and were evaluated for aged creep. The results are shown in Table 3.

TABLE 3
Aged Creep Results

| Adhesive Used | Adhesive Add-on | Laminate Creep |
|---|---|---|
| In Laminate | Levels (gsm) | Values (%) |
| SA-16 | 5.0 | 85 |
| SA-19 | 5.0 | 50 |
| SA-24 | 5.0 | 30 |
| SA-25 | 5.0 | 57 |
| SA-26 | 5.0 | 62 |
| SA-27 | 5.0 | 23 |
| SA-28 | 5.0 | 23 |
| SA-29 | 5.0 | 28 |
| SA-30 | 5.0 | 35 |
| SA-31 | 5.0 | 16 |
| H2840 | 5.0 | 32 |
| SA-16 | 7.5 | 80 |
| SA-19 | 7.5 | 38 |
| SA-24 | 7.5 | 20 |
| SA-25 | 7.5 | 49 |
| SA-26 | 7.5 | 53 |
| SA-27 | 7.5 | 14 |
| SA-28 | 7.5 | 21 |
| SA-29 | 7.5 | 23 |
| SA-30 | 7.5 | 34 |
| SA-31 | 7.5 | 12 |
| H2840 | 7.5 | 28 |
| SA-16 | 10.0 | 73 |
| SA-19 | 10.0 | 32 |
| SA-24 | 10.0 | 16 |
| SA-25 | 10.0 | 41 |
| SA-26 | 10.0 | 47 |
| SA-27 | 10.0 | 9 |
| SA-28 | 10.0 | 11 |
| SA-29 | 10.0 | 15 |
| SA-30 | 10.0 | 22 |
| SA-31 | 10.0 | 7 |
| H2840 | 10.0 | 24 |

As shown above, the inventive adhesives (SA-24, 26, 27, 28, 29, 30, 31) generally exhibited much lower creep than the control adhesives (SA-16, 19, 25 and H2840) at all adhesive levels, except that inventive adhesive SA-26 had relatively high creep and control adhesive H2840 had creep comparable to some of the inventive samples. Notably, SA-26 and SA-30 were made using tackifiers having softening points between 85-89° C. (at the lower end of the suitable range for the invention). The inventive adhesive SA-31, made using a tackifier blend, exhibited the lowest creep at all adhesive levels.

Laminates made using three control adhesive samples and two inventive adhesive samples were prepared and tested using the Static Shear Bond Strength Test described below. The results of the testing are shown in Table 4. While the control laminates failed in less than 5 minutes at the adhesive bond, the inventive laminates did not fail after 8 hours (480 minutes) of testing, due to material failure.

TABLE 4
Static Shear Bond Strength Test at 38° C. (100° F.)

| Adhesive Used | Adhesive Add-on | Time of Bonding | |
|---|---|---|---|
| In Laminate | Levels (gsm) | Failed (minutes) | Failure Mode |
| H2800 | 30 | <1 | Adhesive failure |
| H2800 | 50 | <5 | Adhesive failure |
| H2840 | 50 | <5 | Adhesive failure |
| SA-27 | 30 | >480 | Substrate failure |
| SA-31 | 30 | >480 | Substrate failure |

Laminates including two outer layers of polypropylene spunbond (each having a basis weight of 17 gsm) and an inner layer of LYCRA® 940 elastic strands were formed using a vertical filament laminating process in which the elastic strands were stretched prior to bonding. The laminates were made using the four adhesives indicated in Table 5. In each laminate, the adhesive add-on level was 7.5 gsm, and the adhesive was applied using a melt blowing process.

The four laminates were evaluated for oil resistance using the immersion test described above, wherein each laminate was immersed in mineral oil for 72 hours. The results are reported in Table 5.

TABLE 5
Mineral Oil Resistance Of Vertical Filament Stretch-Bonded Laminates

| Adhesive Used | |
|---|---|
| In Laminate | Observation After 72 Hours |
| H-2840 | Loss of elastic function due |
| | to delamination in less than |
| | one hour |
| SA-24 | No delamination |
| SA-27 | No delamination |
| SA-31 | No delamination |

As shown in Table 5, the inventive adhesives demonstrated oil resistance in the corresponding laminates, whereas the control adhesive (H-2840) did not.

### Test Procedures

Creeping Resistance of Elastic Strands

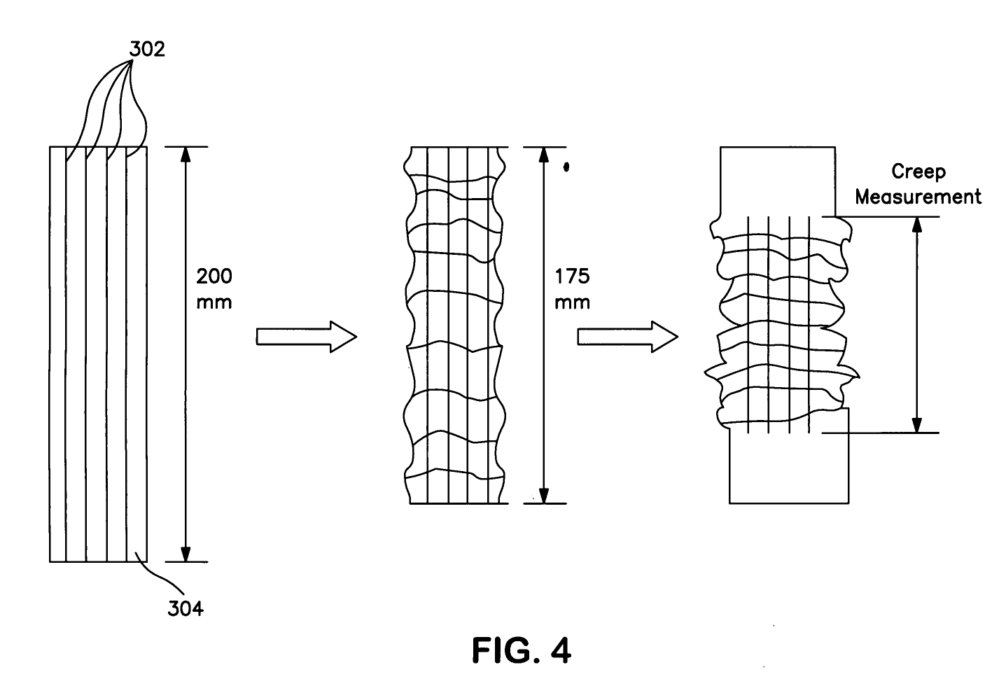

Referring to FIG. 4, twelve elastic strands 302, approximately 2.5 mm apart in the cross-direction and each elongated approximately 250%, were adhesively attached and sandwiched between two 4-inch (10.16 cm) wide continuous polypropylene spunbonded layers 304 to form a laminate. The laminate was fully extended from an initial 57.1 mm length by hanging a weight (about 500 grams or higher) at one end of the laminate, and a 200 mm machine-direction length was then marked. The laminate was then released, such that the 200 mm length snapped back to 175 mm, whereupon the 175 mm length was marked. The laminate was then stapled to a piece of cardboard at the 175 mm length. The marked length of the laminate was then cut to release tension in the elastic strands 302, and the snapback length of the strands was measured.

Initial creep percentage was calculated by first determining the difference between the 175 mm length and the snapback length, then dividing that difference by the difference between the 175 mm length and initial pre-stretched length (57.1 mm) and multiplying the quotient by 100, as shown in the following equation:
Initial Creep %=(175ₘₘ−X_{initial creep})×100/(175−57.1)

The sample was then placed in an oven at 100 degrees Fahrenheit (38° C.) for 90 minutes to measure aging creep. Aging creep percentage was then calculated by determining the difference between the 175 mm length and that snapback length, then dividing that difference by (175 mm−57.1 mm) and multiplying the quotient by 100, as shown in the following equation:
Aging Creep %=(175ₘₘ−Y_{aged creep})×100/(175−57.1)

X_{initial creep} and Y_{aged creep} readings were taken from the averaged measurements of the 24 strands during the tests.

Dynamic Peel Testing

To determine dynamic peel strength, a laminate was tested for the maximum amount of tensile force that was needed to pull apart the layers of the laminate. Values for peel strength were obtained using a specified width of laminate (for the present application, 5.08 cm); clamp jaw width (for the present application, a width greater than 5.08 cm); and a constant rate of extension (for the present application, a rate of extension of 300 millimeters per minute). For samples having a film side, the film side of the specimen is covered with masking tape, or some other suitable material, in order to prevent the film from ripping apart during the test. The masking tape is on only one side of the laminate and so does not contribute to the peel strength of the sample. This test uses two clamps, each clamp having two jaws with each jaw having a facing in contact with the sample, to hold the material in the same plane, usually vertically. The sample size is 2 inches (10.2 cm) wide by 4 inches (20.4 cm). The jaw facing size is 0.5 inch (1.25 cm) high by at least 2 inches (10.2 cm) wide, and the constant rate of extension is 300 mm/min. For a dynamic peel test, one clamp is attached to the top of one substrate of a test panel. The other clamp is attached to the top of the other substrate of a test panel. During testing, the clamps move apart at the specified rate of extension to pull apart the laminate. The sample specimen is pulled apart at 180 degrees angle of separation between the two layers, and the peel strength reported is the maximum tensile strength, in grams, recorded during the test. Each of the peel strengths reported below is an average of five to nine tests. A suitable device for determining the peel strength testing is a SINTECH 2 tester, available from the Sintech Corporation, a business having offices at 1001 Sheldon Dr., Cary, N.C. 27513; or an INSTRON Model TM, available from the Instron Corporation, a business having offices at 2500 Washington St., Canton, Mass. 02021; or the Thwing-Albert Model INTELLECTII available from the Thwing-Albert Instrument Co., a business having offices at 10960 Dutton Rd., Philadelphia, Pa. 19154.

Elongation

The elongation of an elastic composite laminate according to the present invention is suitably determined as follows. A 2.54 cm wide by 10.16 cm long sample of the laminate is provided. The central 3-inch (7.62 cm) area of the sample is marked. The test sample is then stretched to its maximum length, and the distance between the marks is measured and recorded as the “stretched to stop length.” The percent elongation is determined according to the following formula:
{(stretched to stop length (in inches))−3}/3×100
If a 2.54 cm by 10.16 cm area is not available, the largest sample possible (but less than 2.54 cm by 10.16 cm) is used for testing with the method being adjusted accordingly.
Tension Force

The tension force of an elastic composite laminate according to the present invention is determined on a test sample of the laminate having a width of 1 inch (2.54 cm) and a length of 3 inches (7.62 cm). A test apparatus having a fixed clamp and an adjustable clamp is provided. The adjustable clamp is equipped with a strain gauge commercially available from S. A. Mieier Co. under the trade designation Chatillon DFIS2 digital force gauge. The test apparatus can elongate the test sample to a given length. One longitudinal end of the test sample is clamped in the fixed clamp of the test apparatus with the opposite longitudinal end being clamped in the adjustable clamp fitted with the strain gauge. The test sample is elongated to 100 percent of its elongation (as determined by the test method set forth above). The tension force is read from the digital force gauge after 1 minute. At least three samples of the elasticized area are tested in this manner with the results being averaged and reported as grams force per cm width.

Viscosity

To measure viscosity, a material sample is heated to or above 400° F. (204° C.) in a Brookfield Digital Rheometer Model DV-Ill using a Brookfield Temperature Controller (available form Brookfield Engineering Laboratories, a business having offices in Stoughton, Mass.). Spindle #27 was used for all trials and the instrument was appropriately zeroed and calibrated before each test. After the sample is stabilized and sufficiently mixed at 400° F. (204° C.) (or above), readings of the spindle rpm, torque, and viscosity are recorded. The temperature is then lowered, typically in 10° F. (5.55° C.) increments, and the sample allowed to stabilize for 10-15 minutes before subsequent readings of spindle rpm, torque, and viscosity are taken. For purposes of the invention, viscosities can be measured at the temperatures stated in the foregoing specification.

Molecular Weight (Number Average and Weight Average)

Polymer samples can be sent to American Polymer Standard Corp., a business having offices in Philadelphia, Pa., for molecular-weight determinations. The number-average and/or weight-average molecular weights were determined by American Polymer using gel-permeation chromatography on a Waters Model No. 150 gel-permeation chromatograph. The determinations were made using: four, linear, Shodex GPC gel columns; poly(styrene-divinyl benzene) copolymers as standards; trichlorobenzene as the solvent, introduced to the chromatograph at a volumetric flow rate of 1.0 milliliter per minute; an operating temperature of 135° C.; a sample-injection volume of 100 microliters; an M-150C-(64/25) detector; and a GPC PRO 3.13 IBM AT data module.

Static Shear Bond Strength of Laminates

The Static Shear Bond Test was used to determine the approximate time to failure of a laminate in which one substrate was adhesively bonded to another substrate. The test procedure was conducted as follows. Two test panels were obtained from substrates to be adhesively bonded together to form a laminate. One of the test panels was a hook material available from the 3M Corporation under the trade name CS600PIMS. This test panel had a width of 0.75 inches (1.9 cm) and a length of 2 inches (5.08 cm). The other test panel was a stretch-bonded laminate (SBL) including two outer polypropylene spunbond layers each having a basis weight of 17 gsm and elastic strands (formed of a styrene copolymer elastomer blend) having a basis weight of 14 gsm. This test panel had a length of 2 inches (5.08 cm) and a width of 4 inches (10.16 cm). The test panels were laminated together on the spunbond side of the SBL by applying the adhesive across the 5.08 cm length and 1.9 cm of the width of the SBL, and placing the hook material over the adhesive, to form a laminate sample.

The adhesive was applied in a beads or meltblown pattern at an addition level of about 30 or 50 grams per square meter. Prior to its application, the adhesive had been heated to a temperature of about 182° C. (adhesive can be added on the substrates by a machine). After the laminate samples were made and cooled, a 2 kg (4.4 lb) hand roller was then rolled backward and forward over the bonded area for a total of 5 forward and backward cycles. The laminate sample was then suspended vertically in an oven that had been pre-heated to a temperature of about 38° C. A 1000 gram weight was then affixed to the bottom edge of the SBL panel using a clamp or other mechanical securing element.

An automatic time recorder was used to record the failed time. The time at which the SBL panel had detached from the hook panel was recorded. The recorded time corresponded to the approximate time of failure of the laminate sample. The SBL and hook panels were then examined to determine the nature of the failure. If the panels separated such that most of the adhesive remained on one of the panels, then failure was deemed to be an adhesive failure (i.e., failure likely occurred at the interface between one of the test panels and the adhesive composition). If the panels separated such that adhesive remained on both panels, the failure was deemed to be a cohesive failure (i.e., separation likely occurred within the adhesive composition itself). If neither of these conditions arose, but instead one or both of the panels failed, then the failure was deemed a material failure of one or both panels.

The embodiments of the invention disclosed herein are exemplary. Various modifications and improvements can be made without departing from the spirit and scope of the invention. The scope of the invention is indicated by the appended claims, and all changes within the meaning and range of equivalents are intended to be embraced therein.

We claim:

1. An adhesive composition, comprising:
a single-site catalyzed polyolefin-based plastomer having a number average molecular weight of about 1,000 to about 50,000 grams/mol and a crystallinity of 10 to less than 50% by weight;
a crystalline propylene-based polymer having a number average molecular weight of greater than 10,000 to 200,000 grams/mol and a crystallinity of 50-100% by weight;
a tackifier; and
an elastomer.

2. The adhesive composition of claim 1, comprising about 20-70% by weight of the single-site catalyzed polyolefin-based plastomer, about 5-35% by weight of the crystalline propylene-based polymer, about 15-60% by weight of the tackifier, and about 2-20% by weight of the elastomer.

3. The adhesive composition of claim 1, comprising about 25-60% by weight of the single-site catalyzed polyolefin-based plastomer, about 8-30% by weight of the crystalline propylene-based polymer, about 25-55% by weight of the tackifier, and about 3-10% by weight of the elastomer.

4. The adhesive composition of claim 1, wherein the single-site catalyzed polyolefin-based plastomer comprises an ethylene-based copolymer.

5. The adhesive composition of claim 1, wherein the single-site catalyzed polyolefin-based plastomer comprises a propylene-based copolymer.

6. The adhesive composition of claim 1, wherein the crystalline propylene-based polymer has a crystallinity of about 60-85% by weight.

7. The adhesive composition of claim 1, wherein the crystalline propylene-based polymer is selected from the group consisting of a substantially isotactic polypropylene homopolymer, a substantially isotactic propylene-ethylene copolymer, and combinations thereof.

8. The adhesive composition of claim 1, wherein the tackifier comprises a hydrocarbon tackifier having a softening point of at least 90° C.

9. The adhesive composition of claim 1, wherein the elastomer is selected from the group consisting of styrene-isoprene diblock copolymers, styrene-butadiene block copolymers, styrene-isoprene-styrene triblock copolymers, styrene-butadiene-styrene triblock copolymers, styrene-(ethylene-butene)-styrene triblock copolymers, styrene-(ethylene-propylene)-styrene triblock copolymers, styrene-(ethylene-butene)-styrene-(ethylene-butene) tetrablock copolymers, styrene-(ethylene-propylene)-styrene-(ethylene-propylene) tetrablock copolymers, polyisoprenes, polybutadienes, ethylene vinyl acetate copolymers, ethylene methyl acrylate copolymers, ethylene methyl methacrylate copolymers, ethylene ethyl acrylate copolymers, ethylene-n-butyl acrylate copolymers, single-site catalyzed elastomeric copolymers of ethylene and a C₄-C₁₂ alpha-olefin comonomer, and combinations thereof.

10. The adhesive composition of claim 1, wherein the single-site catalyzed polyolefin-based plastomer has a viscosity at 182° C. of at least 3000 centipoise.

11. A laminate, comprising a first facing layer, a second facing layer, and an adhesive composition bonding the first facing layer to the second facing layer, wherein the adhesive composition comprises:
a single-site catalyzed polyolefin-based plastomer having a number average molecular weight of about 1,000 to about 50,000 grams/mol and a crystallinity of 10 to less than 50% by weight;
a crystalline propylene-based polymer having a number average molecular weight of greater than 10,000 to 200,000 grams/mol and a crystallinity of 50-100% by weight;
a tackifier; and
an elastomer.

12. The laminate of claim 11, wherein the first facing layer comprises a polyolefin-based film.

13. The laminate of claim 11, wherein the first facing layer comprises a breathable stretch-thinned film including a polyolefin and a particulate filler.

14. The laminate of claim 11, wherein the first facing layer comprises a plurality of elastomeric strands.

15. The laminate of claim 14, wherein the elastomeric strands comprise polyurethane.

16. The laminate of claim 11, wherein the second facing layer comprises a fibrous nonwoven web.

17. The laminate of claim 11, wherein the second facing layer comprises a polyolefin-based film.

18. The laminate of claim 11, wherein the laminate is oil-resistant.

19. A personal care absorbent article, comprising a chassis defining a waist opening and two leg openings, side panels extending laterally from the chassis, a waist region adjacent to the waist opening and leg regions adjacent to the leg openings, the chassis including a substantially liquid impermeable outer cover, a liquid permeable bodyside liner, and an absorbent core between the outer cover and bodyside liner, wherein at least one of the outer cover, side panels, waist region and leg regions comprises a laminate which includes a first facing layer, a second facing layer, and an adhesive composition bonding the first facing layer to the second facing layer, the adhesive composition comprising:
a single-site catalyzed polyolefin-based plastomer having a number average molecular weight of about 1,000 to about 50,000 grams/mol and a crystallinity of 10 to less than 50% by weight;
a crystalline propylene-based polymer having a number average molecular weight of greater than 10,000 to about 200,000 grams/mol and a crystallinity of 50-100% by weight;
a tackifier; and
an elastomer.

20. The personal care absorbent article of claim 19, wherein the outer cover comprises the laminate.

21. The personal care absorbent article of claim 19, wherein the side panels comprise the laminate.

22. The personal care absorbent article of claim 19, wherein the waist region comprises the laminate.

23. The personal care absorbent article of claim 19, wherein the leg regions comprise the laminate.
